(19) United States
(12) Patent Application Publication
(10) Pub. No.: US 2010/0004700 A1
(43) Pub. Date: Jan. 7, 2010

(54) METHOD OF TREATING TISSUE WITH A SUSPENSON OF TRICALCIUM HYDROXYAPATITE MICROSPHERES

(76) Inventor: Neville Alleyne, La Jolla, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/398,764

(22) Filed: Mar. 5, 2009

Related U.S. Application Data
(60) Provisional application No. 61/034,107, filed on Mar. 5, 2008.

(51) Int. Cl. A61B 17/58; A61M 31/00

(52) U.S. Cl. 606/86 R; 604/500

(57) ABSTRACT

A method of treating an intervertebral disk according to the present invention can include delivering a composition to treat the intervertebral disk. The composition has a plurality of microspheres, and can comprise tricalcium hydroxyapatite microspheres suspended in a sodium carboxymethyl cellulose gel. The composition results in at least one of sealing the defect, increasing a pressure of the disk, increasing a height of the disk, improving stability of the disk and improving structural integrity of the disk.

### CROSS REFERENCE TO RELATED APPLICATIONS

This application claims priority under 35 U.S.C. Section 119(e) to U.S. Provisional Application 61/034,107 filed on Mar. 5, 2008. The entire content of this provisional application is hereby incorporated by reference into this application.

### BACKGROUND OF THE INVENTION

1. Field of the Invention

The present invention relates generally to surgical implants and, more particularly, relates to surgical implants and procedures for repairing spinal disks, ligaments, tendons, and other tissues.

2. Description of the Related Art

Spinal disks comprise a central region called the nucleus pulposus surrounded by a second region known as the annulus fibrosis. The annulus fibrosis portion comprises collagen fibers that may weaken, rupture, or tear, leading to compromised annular confinement of the nucleus and producing disk bulges, herniations and other disk pathologies.

The major causes of persistent, often disabling, back pain are disruption of the spinal disk annulus fibrosis, chronic inflammation of the spinal disk (e.g., herniation), or relative instability of the vertebral bodies surrounding a given spinal disk, such as the instability that often occurs due to a degenerative disease. Spinal disks mainly function to cushion and tether the vertebrae, providing flexibility and stability to the patient's spine. Functionally speaking, spinal disks comprise a central hydrostatic cushion, the nucleus pulposus, surrounded by a containing multi-layered ligament, the annulus fibrosis. As spinal disks degenerate, they can, for example, lose their water content and height which brings the vertebrae closer together. This phenomena results in a weakening of the shock absorption properties of the spinal disk and a narrowing of the nerve openings in the sides of the spine which may pinch the nerve. This spinal disk degeneration can eventually cause back and leg pain. Weakness in the annulus fibrosis from degenerative spinal disks, or from spinal disk injury, can allow fragments of nucleus pulposus within the spinal disk space to migrate into the spinal canal. There, displaced nucleus pulposus or protrusion of annulus fibrosis, e.g., herniation, may impinge on spinal nerves. The mere proximity of the nucleus pulposus or a damaged annulus fibrosis to a nerve can cause direct pressure against the nerve, resulting in numbness and weakness of leg muscles.

It is estimated that approximately 80% of the population at some time in their life suffer back injuries necessitating consultation from a medical doctor for treatment of back pain. A good portion of these back injuries are related to spinal disk protrusions or herniations, and a smaller percentage are related to internal disk derangement.

Often, inflammation from spinal disk protrusions or herniations can be treated successfully by non-surgical means, such as rest, therapeutic exercise, oral anti-inflammatory medications or epidural injection of corticosteroids. In some cases, the spinal disk tissue is irreparably damaged, thereby necessitating removal of a portion of the spinal disk or the entire spinal disk to eliminate the source of inflammation and pressure. At the present time, a procedure which is performed as an open procedure is called a microdiskectomy in which small midline incision is made in the lumbar spine with the dissection being carried down to the lamina. The lamina is then prepared with a keyhole laminotomy, and the ligamentum flavum is then removed. Once this occurs the cal sac and nerve root are retracted to the contralateral side, thus providing exposure to the disk space. Intraoperative lateral x-ray can be implemented to confirm the position of the disk, and direct visualization of the herniation can be noted. Typically, a 15-blade is used to make an annulotomy, and the removal of the herniated fragment or fragments is then undertaken. In some cases this completes the disk excision procedure, while in other cases the surgeon actually places a pituitary rongeur or ring curette into the disk space and removes additional disk material, but a subtotal diskectomy is performed. At the conclusion of the surgery, the offending disk fragment or fragments has been removed, and there is now an annular defect that varies in size. This defect may be as small as 0.5 mm×0.5 mm and as large as 10 mm×15 mm. Typical annulotomies, however, are approximately 5 mm×5 mm. Many current technologies fail to offer surgeons plugs or other materials to place into the disk space for preventing recurrent disk herniation.

In some cases, a tear in the annulus fibrosis may begin the process of disk degeneration. In removing the protruded disk tissue and entering into the annulus to remove loose material, traditional spinal surgery may accelerate the disk degeneration and potentially decrease the shock-absorbing capacity of the spinal disk.

Other techniques to treat disk disease include implanting a fusion device into the disk space to stabilize the motion segment. Fusion techniques involve radical subtotal discectomies, however, and involve both anterior and posterolateral stripping of muscles that require lengthy periods of immobilization during recovery. Other treatment options include disk arthroplasty, which presently requires anterior resection of the anterior longitudinal ligament and placement of motion preservation devices to simulate motion of the degenerated disk. These treatments are lengthy, costly, and associated with significant morbidity and mortality intraoperatively and postoperatively.

To overcome the disadvantages of traditional traumatic spine surgery, minimally invasive spine surgery was developed. Endoscopic spinal procedures, for example, are less invasive than open spinal procedures. In an encloseopic procedure, the spinal canal may not be violated and therefore epidural bleeding with ensuring scarring may be minimized or avoided. In addition, the risk of instability from ligament and bone removal is generally lower in endoscopic procedures than with open diskectomy. Further, more rapid rehabilitation facilitates faster recovery and return to work. Minimally invasive techniques for the treatment of spinal diseases or disorders include diskography, chemonucleolysis, laser techniques and mechanical techniques. These procedures generally require the surgeon to form a passage or operating corridor from the external surface of the patient to the spinal disk(s) for passage of surgical instruments, implants, and the like. Typically, the formation of this operating corridor requires the removal of soft tissue, muscle, or other types of tissue depending on the procedure (e.g., laparascopic, thoracoscopic, arthroscopic, back, etc.). Once the operating corridor is established, the nerve root may be retracted and a portion or all of the spinal disk removed. Following removal, typical techniques do not implement an annular sealant or other means to efficiently and effectively treat the annular defect or opening to minimize the possibility of recurrent complications such as, for example, future nuclear herniations. Thus, effective repair of spinal defects while minimizing recurrent complications remains a significant aspect in the treatment of spinal diseases.

Various forms of tissue repairs are also performed on ligaments and tendons, which provide support and stability to the musculoskeletal system. Generally consisting of bands or sheets of fibrous connective tissue, ligaments and tendons when damaged can be painfull and often times debilitating. Treatments of these connective tissues can comprise repair by means such as suturing, or can comprise complete or partial replacement with other biological or synthetic materials. As a result of the complexity and functionality of these tissues and general repair considerations, it is generally preferred that the treatment retain and return these tissues to their pre-damaged conditions.

Regarding the repair of ligaments and tendons, as distinguished from replacement, one of the more common ligament repair procedures involves reconstruction of the anterior cruciate ligament (ACL). Several hundred thousand ACL repairs and reconstructions are performed every year in the United States. That number continues to grow as the population continues to become more active in recreational sports and competitive sports such as soccer, football, basketball, and track and field. A segment of the population comprises individuals who will end up sustaining partial injuries to their ACL or posterior cruciate ligaments (PCL), requiring surgery to assist in the healing. It has been known for some time that ACL deficient knees and PCL deficient knees can subsequently lead to other intraarticular pathologies, such as meniscal tears or collateral ligament attenuations. Consequently, as with injuries to other ligaments and tendons, the pursuit of effective interventions for efficiently treating injured ligaments and tendons continues to be an active and needed field of endeavor.

### SUMMARY OF THE INVENTION

In accordance with one aspect of the present invention, methods are provided for treating tissues using a composition comprising tricalcium hydroxyapatite microspheres suspended in a sodium carboxymethyl cellulose gel.

In one embodiment, a method of treating an intervertebral disk comprises performing a discography procedure and delivering a composition of tricalcium hydroxyapatite microspheres suspended in a sodium carboxymethyl cellulose gel to the invertebral disk through an aperture present in the spinal disk created during the discography procedure.

In another embodiment, a method of treating a ligament or tendon comprises identifying a defect in the ligament or tendon and delivering a composition of tricalcium hydroxyapatite microspheres suspended in a sodium carboxymethyl cellulose gel into the tissue of the ligament or tendon.

In another embodiment, a method of treating a fascial tissue comprises identifying an inguinal, umbilical, or abdominal wall defect and delivering a composition of tricalcium hydroxyapatite microspheres suspended in a sodium carboxymethyl cellulose gel into the fascial tissue in the region of the defect.

In addition, a method of treating an intervertebral disk comprises inserting one or more needles into the intervertebral disk creating one or more apertures in the intervertebral disk; injecting contrasting die into the intervertebral disk; and delivering a composition of tricalcium hydroxyapatite microspheres suspended in a sodium carboxymethyl cellulose gel to the intervertebral disk through at least one of said one or more apertures.

### DETAILED DESCRIPTION OF THE INVENTION

Any feature or combination of features described herein are included within the scope of the present invention provided that the features included in any such combination are not mutually inconsistent as will be apparent from the context, this description, and the knowledge of one skilled in the art. In addition, any feature or combination of features may be specifically excluded from any embodiment of the present invention. For purposes of summarizing the present invention, certain aspects, advantages and novel features of the present invention are described herein. Of course, it is to be understood that not necessarily all such aspects, advantages or features will be embodied in any particular embodiment of the present invention.

In reference to the disclosure herein, for purposes of convenience and clarity only, directional terms, such as, top, bottom, left, right, up, down, upper, lower, over, above, below, beneath, rear, and front, may be used. Such directional terms should not be construed to limit the scope of the invention in any manner. It is to be understood that embodiments presented herein are by way of example and not by way of limitation. The intent of the following detailed description, although discussing exemplary embodiments, is to be construed to cover all modifications, alternatives, and equivalents of the embodiments as may fall within the spirit and scope of the invention.

The present invention provides compositions and methods for selectively treating defects within or on a spinal disk. These procedures include laminectomy/diskectomy procedures for treating herniated spinal disks, decompressive laminectomy for stenosis in the lumbosacral and cervical spine, medial facetectomy, posterior lumbosacral and cervical spine fusions, treatment of scoliosis associated with vertebral disease, foraminotomies to remove the roof of the intervertebral foramina to relieve nerve root compression and anterior cervical and lumbar discectomies. These procedures may be performed through open procedures (e.g., laminotomy, laminectomy, hemilaminotomy and hemilaminectomy), or using minimally invasive techniques, such as thoracoscopy, arthroscopy, laparascopy, diskogrophy (e.g., performed percutaneously through a posterior, posterolateral, lateral, anterior or anterolateral approach to the spinal disk) or the like.

According to one embodiment of the present invention, a composition comprising microspheres suspended in a cellulose gel is delivered to a tissue to treat a defect in the tissue. In one embodiment, a composition comprising calcium hydroxyapatite microspheres suspended in a sodium carboxymethyl cellulose gel is delivered to an intervertebral disk to treat a defect in the disk. According to one embodiment, the composition may comprise about 30% v tricalcium hydroxyapatite microspheres ranging in size from approximately 25 to 40 micron diameter. The remaining 70% v may comprise a solution of carboxymethyl cellulose gel. A commercially available material of this type is Radiesse®, a composition comprising a suspension of about 30% tricalcium hydroxyapatite microspheres of approximately 25-40 micron size in a carboxymethyl cellulose gel.

Compositions in accordance with the present invention can be used, for example, to stabilize mammalian intervertebral disks. Nutrients are provided to the intervertebral disk through the very delicate endplates above and below the disk. The process of imbibition and/or solutransport allows for the nutrients to reach the disk and provide hydration and strength to this unique structure. Such infiltration occurs much more slowly in the intervertebral disk, especially in a nearly intact outer annulus. Without being bound to any particular theory, it is believed that the tricalcium hydroxyapatite particles of one embodiment of the present invention provide a matrix for local cellular infiltration into the intervertebral disk. In another embodiment, tricalcium hydroxyapatite microspheres suspended in a sodium carboxymethyl cellulose gel provide augmentation to the annulus via minimally invasive or percutaneous delivery methods.

According to one implementation of the present invention, a condition known as internal disk derangement or annular fissures can on occasion be detected using magnetic resonance imaging (MRI), but in certain instances may more readily be discerned using computed tomography (CT) diskography. These annular fissures or tears can lead to persistence in back pain, and eventually can lead to frank herniations and/or lumbar segmental instability. Such changes that may be seen on MRI are sometimes further evaluated with provocative CT diskography which reveals the location of the annular tear or tears. Procedures such as IDET (intradiscal electrothermal annuloplasty) and nucleoplasty have become more prevalent. Use of compositions as described herein in these contexts can entail insertion by the interventional radiologist, anesthesiologist, physiatrist or surgeon to facilitate the sealing or other treating of the annular tear from the inside out of the spinal disk.

In accordance with an aspect of the present invention, a composition of tricalcium hydroxyapatite microspheres suspended in a sodium carboxymethyl cellulose gel is provided for sealing tears or other defects or conditions of a spinal disk, such as a rent in the annulus fibrosis of a spinal disk. The composition can be inserted into a ruptured spinal disk, filling a portion of the nucleus pulposus and/or annulus fibrosis and providing a seal. In one implementation, the composition is inserted into a center region of the ruptured spinal disk. According to certain aspects, the composition is inserted into the nucleus pulposus after a microdiscectomy which closes the iatrogenic rent or annulotomy that the surgeon creates, thereby minimizing the risk for recurrent herniation, or is administered as an injectable sealant into the center of the spinal disk, for example, after a diskography procedure in order to seal one or more annular tears.

Compositions of the present invention are not limited to containing solely tricalcium hydroxyapatite microspheres suspended in a sodium carboxymethyl cellulose gel. Other materials that have been used as dermal fillers can be used in combination with the tricalcium hydroxyapatite microspheres and sodium carboxymethyl cellulose gel. Such added compositions include conventional derma fillers, including but not limited to: Restylane®, a composition comprising 2% cross-linked hyaluronic acid produced biotechnologically from streptococcus equi; Artecoll®, a composition comprising a suspension of 20% 40 micron PMMA (polymethylmethacrylate) microspheres in a bovine collagen solution; PMS 350 medical grade silicone fluid (dimethylpolysiloxane) of 350 centistoke viscosity; New-Fill, a composition comprising approximately 4.5% poly-L-lactic acid microspheres of 2-50 micron size suspended in 2.7% methylcellulose; Revidemm® Intra, a composition comprising a suspension of 2.5% dextran microspheres, 40 micron Sephadex, and 2% hyaluronic acid, 2.5 MDa of bacterial origin—Rofilan; Dermalive®, a composition comprising a suspension of hydroxymethyl methacrylate fragments in 1.14% cross-linked hyaluronic acid of bacteriologic origin; Aquamid®, a clear 5% cross-linked gel comprising polyacrylamide; Evolution, a composition comprising a suspension of 6% polyvinylhidroxide microspheres of 5-80 micron size in a 2.5% polyacrylamide gel; and To the extent such tears or defects are treated using the present invention, risks for recurrent spinal disk herniations and possible revision surgeries can be attenuated or eliminated. Such revisions typically entail slightly larger incisions, greater bony resection, removal of scar tissue, more difficult retraction, increased bleeding, increased anesthetic time, and increased risk for battered nerve roots or possible injury to the dura or root sleeves resulting in potential Cerebro-Spinal Fluid (CSF) leak, fistula, infection, etc. As a result of the minimized need for revision surgery, surgical outcome can be improved and the need for repeat surgery at the same level can be decreased.

Moreover, with the perhaps increased use of provocative diskography to ascertain, for example, whether adjacent segments above or below a planned fusion need to be incorporated, a user can instill the composition to minimize the extension of the fusion to the adjacent segment. Using conventional procedures, for example, if an unstable motion segment were planned to be fused and preoperative provocative diskography revealed the adjacent segment (e.g., the adjacent spinal disk) as also being symptomatic, that level would be included in the fusion mass. However, in accordance with an aspect of the present invention, the composition of the present invention can be instilled into the adjacent segment prior to the surgery to help seal the annular tear or tears. In one implementation, the composition of the present invention can be instilled into the adjacent segment during the preoperative provocative diskography. As a result, the use of the composition is not limited to microdiscectomy or open diskectomy procedures, but can also be used for closed procedures in which, for example, imaging studies have proven that there are annular tears or rents which reproduce concordant pain. Delivery of the composition, in accordance with one implementation of the present invention, may be especially suited for annular tears which are not asymptomatic and which do not produce discordant pain.

Implantation of the composition, if performed in the context of a closed procedure, can be accomplished from a posterior midline or posterolateral approach or a direct lateral approach. If performed in the context of an open procedure, delivery of the composition can be achieved from a posterior midline approach, posterolateral approach, anterior, anterolateral, or direct lateral approach. It is therefore possible that if an anterior approach is being utilized for an anterior diskectomy alone, the composition of the present invention can be instilled through a syringe and needle into that nucleus pulposus space after, for example, an offending spinal disk fragment or fragments have been removed. In certain implementations, the material can be introduced via flexible catheters of variable length and diameter, such as, for example, standard percutaneous needles and standard catheter tips known in the industry. In an exemplary open procedure where for example a laminectomy or microdiscectomy is being performed, it may be easier to deliver the composition as used according to the present invention with the aid of an injection syringe, such as a 25-gauge syringe with a 3 or 4″ needle.

The maturation of the biocompatible alloplastic implant of the present invention, in accordance with an aspect of the present invention, can over time afford additional, or at least partial, stabilization to the annulus fibrosis which can then provide additional support to the motion segment involved. This change in the biomechanics can translate into a partial increase in the stability for this motion segment. Having an annular tear generally can cause a weakening in the supporting structure of the motion segment. Treating the nucleus pulposus of a spinal disk with the composition of the present invention can in certain implementations allow a maximum amount of the nuclear material to remain centrally located and/or can increase the integrity of the surrounding annular fibers.

Compositions according to the present invention can be injected into the injured disk percutaneously or through minimally invasive surgery at the early stages of degeneration to retard the degenerative process. Annular tears can propagate and lead to disk herniations and loss in disk height. The degenerative changes may occur not only in the disk, but in the facets and associated osteophytes, leading to decreased range of motion in the involved motion segment. Delivering the composition of the present invention early in the degenerative process can help stabilize annular tears, assist the body's reparative mechanisms, and decelerate the degenerative process.

Compositions delivered via minimally invasive or percutaneous methods according to aspects of the present invention offer the advantage of allowing the outer annulus to remain intact, while the inner portion of the nucleus and nuclear annular border become stable. Without being bound to any particular theory, it is believed that compositions according to the present invention stabilize the intervertebral disk by aiding the transfer of nutrients to the disk through imbibition or solutransport.

The compositions of the present invention can comprise a plurality of microparticles, such as solid microparticles in representative embodiments. In modified implementations, the microparticles may not be altogether solid, such as implementations involving hollow or porous microparticles. As used herein, the term “microparticles” refers to microparticles (e.g., in a dust or powder form) possessing an average diameter of 500 microns or less. Typically, the average diameter will be greater than about 20 microns rendering the microparticles too large to be “eaten” by monocytes. The microparticles can have diameters sufficient to keep them from being washed away through lymph tracts or other tissue tracts from the implantation site. If the microparticles do not have a spherical form, then the diameter as used herein refers to the greatest diameter of the smallest cross sectional area. It is, however, also possible to use smaller microparticles ranging from 4 to 5 microns or 5 to 10 microns in diameter. Typically, the microparticles will have an average diameter less than about 200 microns. In representative embodiments, the microparticles can have an average diameter of about 15 to about 200 microns and in certain implementations from about 25 to about 40 microns. In representative configurations, the microparticles are small enough to be injected through a fine gauge cannula (e.g., 25 gauge) or an injection syringe to the desired spinal disk region. Particles having the diameters specified herein may have a relatively minimal effect on the surrounding tissues, i.e., the dura of the cal sac or nerve root sleeves.

Due to the formed surface and size of the microparticles used, they are not detected by the endogenous macrophages as foreign bodies so that no defensive reaction takes place. According to a representative embodiment, the microparticles have spherical forms or spherical-like forms capable of forming closely-packed arrangements at the site where they have been implanted and further capable of being individually encapsulated by the scar tissue.

During a conventional provocative CT diskography, opening spinal-disk pressures are often measured. In the context of diskography, or any of the above-mentioned procedures, it is possible in accordance with certain aspects of the present invention for a spinal-disk opening pressure to be significantly altered by the introduction of the biocompatible alloplastic implant into the nucleus pulposus of that spinal disk and, preferably, into a central region of the nucleus pulposus, so that for example, at least partial sealing of the spinal disk can be effectuated from the inside out.

As a result of delivery of the composition into a spinal disk, a seal or occlusion can be formed in the annulus fibrosis defect via, for example, in one implementation, displacement of nucleus pulposus from the site of implantation (e.g., an intermediate or, more preferably in some embodiments, central region of the nucleus pulposus) in a direction toward, for example, an annulus fibrosis defect, so that nucleus pulposus is displaced into a vicinity of the annulus fibrosis defect thus serving to strengthen or otherwise affect at least one property of the spinal disk or defect. In another implementation of the present invention, a seal or occlusion can be formed in the annulus fibrosis defect via, for example, introduction of the composition into the nucleus pulposus in a direct or proximate vicinity of the annulus fibrosis defect thus serving to enhance or otherwise affect at least one property of the spinal disk or defect. For instance, if the composition is injected or inserted in either a closed fashion or an open fashion, and if a sufficient portion of the composition is placed (and/or caused to solidify or mature) in the center, increased nuclear support can ensue giving rise to not only an increased annular integrity but also, for example, an increased nuclear stability.

The microspheres, which in a representative embodiment may comprise tricalcium hydroxyapatite, after being inserted into the spinal disk space, may be encapsulated by delicate capsules of connective tissue and/or are embedded into connective-tissue tissue or fibers and remain stationary in the tissue.

Once placed into the nucleus pulposus, the composition may mimic or provide a substitute for at least one characteristic of the physiologic structure of the spinal disk. For example, the composition may mimic the spinal disk and operate as a partial artificial disk or operate as a partial artificial nucleus pulposus. Accordingly, a morphology of a disco gram may be improved following delivery of the composition. For instance, the accumulation of the microparticles of the composition and/or the accumulation of scar tissue around the microparticles within the nucleus pulposus can impart a certain physical stability to the interior of the spinal disk and/or to exterior portion of the annulus fibrosis. Later testing after the sealant (i.e., the composition) has matured (e.g., been incorporated into the host tissue through, for example, formation of permanent scar tissue around the microparticles of the composition) can yield an increase in the pressure gradient of the nucleus pulposus. Also, a slight increase in spinal disk space height may be achieved in proportion to the amount of the composition instilled which may vary from spinal disk to spinal disk, but which in a representative embodiment does not exceed about 3 to 4 cubic centimeters (ccs) and, typically, is within a range of about 0.5 to 1.5 ccs. During injection according to one embodiment, it is advantageous to release pressure on the syringe plunger when the tip of the needle is within about 3-5 mm from the outer surface of the disk during removal of the needle from the disk.

Regarding maturation of the microspheres, as a result of the size and physical stability of the tricalcium hydroxyapatite microspheres according to one embodiment, they cannot be phagocytised or lysed. In order to isolate the foreign body, the animal body can only fibrotically wall off the foreign bodies in the form of scar tissue. Such a process takes place with almost any foreign body which cannot be destroyed by the animal body. Prior to or substantially commensurate in time with delivery of the composition and any removal of a part of the spinal disk (if applicable), the annular fibers that are attached to the vertebra end plates above and below can be minimally resected to allow punctate bleeding to occur from, for example, the edges of the end plate.

To the extent present, the fibrotic growth of connective tissue is a natural reaction to the lesion of the tissue caused by the injection cannula and to the presence of the microparticles. The fibrotic reaction may occur during 3-6 months after delivery of the composition due to the smooth and chemically inert surfaces of the microparticles (e.g., tricalcium hydroxyapatite microspheres). From then on, the microspheres remain in the tissue without reaction and provide for the formation and existence of permanent fibrovascular connective tissue.

The composition can in one implementation comprise a histocompatible solid in the form of a powder. The microparticles forming the solid may be incorporated into a suspending agent and injected, for instance, with an injection needle at the desired spinal disk level.

Although not always necessary, it can be advantageous for the microparticles used according to an embodiment of the present invention to have a smooth surface and be free from corners and edges, such that the microparticles do not have sharp transitions on their surfaces. In addition they may not have peaks of any kind or tapered projections. According to one implementation, the surface does not have pores. In another implementation, the surfaces may comprise pores. Although smooth, and especially spherical particles can be advantageous, in some embodiments, non-smooth microparticles of with corners or peaks or the like may still be used in the present spinal disk treatment application.

In many advantageous embodiments, the transition from one outer surface to the other outer surface of the microparticles as used according to the present invention occurs in a continuous manner. If such transitions are present as is the case for the edges of a cube, such transitions may be smoothed. According to an embodiment of the present invention, microparticles which are crystalline (for instance needle-shaped) or microparticles which have been obtained by mechanically breaking up greater units into small pieces, are not used to the extent the microparticles possess the above-mentioned sharp edges and corners. Due to the smooth surface structure damage to cells and other tissue structures is minimized. In addition, the danger of causing reactions of the tissue, such a foreign body reactions or granulous formation, which may be followed by infections, is minimized.

In one implementation, dynamically balanced microparticles and in particular microparticles having an elliptic or spherical form can be used. In addition, it is possible to use microparticles of a different geometrical form if all, or in another embodiment, a majority, of the microparticles have a smooth and smoothed-off surface.

The mixing ratio of the components of the suspending agent can be chosen according to the needs, and in particular according to the size of the syringe used to deliver the composition. For the application or injection of the microparticles used according to an embodiment of the present invention, the microparticles can be suspended or slurried in a fluid inert medium. In one particular implementation, a ratio of two volume parts of the suspending agent and one volume part of the microspheres is chosen.

It will be appreciated that the invention has a variety of aspects. In accordance with some of these aspects, a composition comprising tricalcium hydroxyapatite microspheres can be utilized for annular welding or sealing of a spinal disk defect, such as a ruptured spinal disk. The composition can include solid microparticles which have smooth surfaces that are substantially free from corners and edges and which can in certain implementations be suspended in a biocompatible medium, such as carboxymethyl cellulose. The composition can be inserted into a ruptured spinal disk, filling a portion of the nucleus pulposus or annulus fibrosis and providing a seal. In one implementation, the composition is inserted into a central region of the ruptured spinal disk. Insertion of the composition into the ruptured spinal disk can attenuate a risk for recurrent spinal disk herniation and restore at least a portion of a structural integrity or shock absorbing capacity of the spinal disk.

A method of treating an intervertebral disk according to the present invention can comprise performing a discography procedure and delivering a composition of tricalcium hydroxyapatite microspheres suspended in a sodium carboxymethyl cellulose gel to the intervertebral disk through at least one aperture present in the disk created during the discography procedure. The method can further comprise identifying an annular tear in an intervertebral disk, and delivering the composition to the intervertebral disk in the region of the annular tear. In one embodiment, the composition stabilizes a tear in the outer annulus of the intervertebral disk. In another embodiment, the composition fills at least a portion of the nuclear annular border.

The method according to the present invention can further comprise identifying a defect. The identifying of a defect can comprise, for example, identifying a defect through a scope. In typical implementations, the identifying of a defect can comprise identifying a focal outpouching comprising a displacement of nucleus pulposus within a partially torn or thinned annulus fibrosis of the spinal disk, can comprise identifying an extrusion comprising displaced nucleus pulposus which remains in continuity with an interior of the spinal disk through a rent in an annulus fibrosis of the spinal disk, or can comprise identifying a sequestration comprising displaced nucleus pulposus which does not remain in continuity with an interior of the spinal disk.

Delivering the composition according to aspects of the present invention can comprise injecting the composition into the spinal disk while viewing at least a part of the spinal disk through a scope. The scope can comprise a video fluoroscope, and the inserting can be fluoroscopically guided. In one implementation, the composition can comprise a water soluble radiopaque dye to facilitate visualization during delivery of the composition into the spinal disk. The radiopaque dye can comprise barium or metrizamide. In a typical implementation, the inserting can comprise inserting about 3 or 4 cubic centimeters (ccs) or less of the composition into a nucleus pulposus of the spinal disk, and in certain implementations the inserting comprises inserting about 0.5 to 1.5 cubic centimeters (ces) of the composition into the nucleus pulposus of the spinal disk.

The inserting may be followed by a height of the spinal disk being increased, wherein the increase in height is proportional to an amount of the composition inserted into the spinal disk. In accordance with one aspect of the present invention, the inserting may be followed by a structural integrity of the spinal disk being improved, compared to a structural integrity of the spinal disk before the inserting. For example, a stability of the annulus fibrosis of the spinal disk may be improved relative to a stability of the annulus fibrosis before the inserting, whereby a biomechanical property of a motion segment of the spinal disk is improved compared to biomechanical property of the motion segment before the inserting. In one embodiment of the present invention, a method of treating an intervertebral disk using compositions described herein includes one of stabilizing an annular tear or other disk defect, sealing a disk defect, reducing loss in the height of the disk, preserving the height of the disk, increasing the height of the disk, increasing a pressure of the disk, improving stability of the disk, and improving structural integrity of the disk.

When the spinal disk is juxtapositioned in proximity to at least one of an upper vertebra and a lower vertebra, at least one aperture can be formed in an endplate of one or both of the upper vertebra and the lower vertebra. Typically, the spinal disk is juxtapositioned between an upper vertebra and a lower vertebra, and a plurality of apertures are formed in an endplate or endplates of at least one of the upper vertebra and the lower vertebra. The aperture or apertures can be formed using a needle, which may already be present in the spinal disk during an ongoing procedure such as, for example, a diskography procedure.

In representative implementations of the methods disclosed herein, the defect comprises a spinal annular defect. For instance, the defect can comprise an internal disk derangement. Insertion of the composition into the spinal disk can cause a seal to be formed in and around the spinal annular defect. This seal can create a more stable motion segment of the spinal disk compared to a motion segment of the spinal disk before the inserting, by for example imparting increased stability to the spinal disk relative to a stability of the spinal disk before the inserting.

In one embodiment of the present invention, the composition is injected through a syringe via a discogram or a CT-guided needle syringe. The injectionist (such as an orthopedic surgeon, neurosurgeon, physiatrist, pain management doctor, anesthesiologist, interventional radiologist) can visualize an annular tear on discography using a routine CT, CT myelography, MRI, ultrasound, or most preferably, pressurized CT discography. During a pressurized CT discography according to one embodiment, the disk is injected with barium, metrizamide, or a radial opaque dye to better outline the annular tear in the pressurized disk. The outline of the discogram can allow the injectionist to ascertain concordant or discordant pain and give a specific morphology of the tear within the disk.

Thus, according to one embodiment, the inserting can be performed during a diskography procedure, and the defect can comprise at least one annular rent. During the diskography procedure, the identifying can comprise an initial visualization of the at least one rent followed by the inserting being performed during the same diskography procedure. In accordance with one implementation of the inventive methods disclosed herein, the diskography procedure comprises a provocative diskography procedure wherein the identifying comprises an initial visualization of the at least one rent and wherein the inserting is performed during the same provocative diskography procedure.

According to another implementation, a method of treating an intervertebral disk comprises inserting one or more needles into the intervertebral disk creating one or more apertures in the intervertebral disk; injecting contrasting die into the intervertebral disk; and delivering a composition comprising tricalcium hydroxyapatite microspheres suspended in a sodium carboxymethyl cellulose gel to the intervertebral disk through at least one of the apertures.

According to yet another implementation, the diskography procedure can be performed percutaneously through one of a posterior, posterolateral, lateral, anterior or anterolateral approach to the spinal disk.

In other implementations, the inserting can be performed during an open procedure, and can comprise inserting the composition using a syringe and needle into the spinal disk in one of a laminotomy, laminectomy, hemilaminotomy and hemilaminectomy open procedure.

Another method of the present invention that can be performed on a spinal disk includes delivering a composition comprising tricalcium hydroxyapatite microspheres suspended in a sodium carboxymethyl cellulose gel into a spinal disk. The delivering can be preceded by inserting an injection device into the spinal disk, and the composition can be delivered though the injection device and into the spinal disk. When the spinal disk is positioned in proximity to at least one of an upper vertebra endplate and a lower vertebra endplate, the method can comprise forming one or more apertures or perforations in at least one of the upper vertebra endplate and the lower vertebra endplate.

Delivering the composition can comprise delivering tricalcium hydroxyapatite microspheres suspended in a sodium carboxymethyl cellulose gel into a nucleus pulposus of the spinal disk, such as an central or non-perimeter region of the spinal disk. The delivering can be preceded by detecting a condition in the spinal disk, and the composition can be delivered into the spinal disk to treat the condition. Moreover, the detecting of a condition can comprise detecting a displacement of inner disk material within a partially tom or thinned annulus of the spinal disk, and the delivering can comprise delivering an amount on the order of about 3 to 4 cubic centimeters (ccs) or less of the composition into the spinal disk.

It may also be noted that the techniques described herein can be used to advantageous effect for treating household pets such as dogs and cats. In these cases, vertebral fusions and similar procedures are often cost prohibitive, so any lower cost techniques for disk repair would be beneficial.

According to another embodiment of the present invention, a composition comprising microspheres suspended in a cellulose gel is delivered to a tissue to treat a defect in a ligament or tendon. In one embodiment a method of treating a ligament or tendon comprises identifying a defect in the ligament or tendon and delivering a composition comprising tricalcium hydroxyapatite microspheres suspended in a sodium carboxymethyl cellulose gel into the tissue of the ligament or tendon. Compositions of the present invention are not limited, however, to tricalcium hydroxyapatite microspheres suspended in a sodium carboxymethyl cellulose gel. Various compositions such as those described in greater detail above can be used to repair tissue defects in ligaments and tendons using method described herein.

Ligaments and tendons that can be repaired or augmented according to embodiments of the present invention include but are not limited to the Achilles tendon, rotator cuff tendon, quadriceps tendon, triceps tendon, anterior bruciate ligament, posterior cruciate ligament, medial collateral ligament, lateral collateral ligament, hip capsule, meniscus, anterior talofibular ligament calcaneofibular ligament, posterior talofibular ligament, deltoid ligament, spring ligament, intrametatarsal and intermetatarsal ligaments, metatarsophalangeal ligaments, sacroiliac ligaments, iliolumbar ligaments, inguinal ligaments, superspinous ligaments, interspinous ligaments, facet capsular ligaments, anterior longitudinal ligament, posterior longitudinal ligament, ligamentum flavum, atlantdaxial ligaments, alar ligaments, anterior glenohumeral ligaments, posterior glenohumeral ligaments, acromioclavicular ligaments, coracoclavicular ligaments, coracoacromial ligaments, stemoclavicular ligaments, costotransverse ligaments, suprascapular ligament, the trapezoid ligament, conoid ligament, coracohumeral ligament, annular ligament of the elbow, ulnar collateral ligament of the elbow, radial collateral ligament of the elbow, palmar radiocarpal ligament, palmar ulnocarpal ligament, dorsal ulnocarpal ligament, radial collateral ligament, the dorsal radiocarpal ligament, radial collateral ligaments of the carpus, the palmar carpometacarpal ligaments, the palmar metacarpal ligaments, pisometacarpal ligament, pisohamate ligament, the dorsal metacarpal ligaments, the dorsal carpometacarpal ligaments, the dorsal ulnocarpal ligament, the dorsal radioulnar ligament, the metacarpophalangealarcuate complex ligament, and Poupart ligament. The scope and field of the present invention for orthopedic tendons and ligament reconstruction, however, is vast and is intended to include any of the major or minor joints with ligaments or tendons that may be injured or otherwise determined to be in need of or likely to benefit from an intervention or treatment using compositions described herein.

Generally, in one or more of any of the above applications, the addition of the tricalcium hydroxyapatite microspheres suspended in a sodium carboxymethyl cellulose gel may allow increased structural support and integrity to the repair site (e.g., the motion segment). Accordingly, representative applications of the compositions can include providing structural support to ligaments or tendons that are partially or completely severed, or can include augmenting the repairs. For instance, the composition can provide a biological scaffold helping to support fixation for repair or augmentation of the ligament or tendon in question, and can also operate as a partial permanent connective tissue scaffold in the ligament or tendon repair.

As but one exemplary area of application of the present invention, the composition can be inserted into either the ACL or PCL requiring surgery to thereby assist in the healing. The tissue-generating implant can be used, for example, to strengthen the collateral ligaments in the event that they are torn with a grade 1 or grade 2 tear to thereby potentiate or accelerate the healing, and in some instances obviate the need for, or reduce a necessity or extent of, surgical intervention.

Treatment of ligaments or tendons by insertion of the compositions in accordance with the present invention can serve, ultimately, to augment those ligaments or tendons with additional host tissue. The additional host tissue is not implanted but, rather, is generated naturally by the host at the site of the insertion, and integrated into existing tissues by the host at the site of insertion. This natural introduction of host tissue onto or into the ligament or tendon can increase the healing strength of these tissues.

Following insertion (e.g., injection) of the composition into an area or areas of interest of a ligament or tendon, such as an ACL, PCL or one of the medial and collateral ligaments, the composition may be both resorbed and replaced with host tissues. Optionally, in any of the above-stated applications, such as injection of the composition into the collateral ligaments, ligaments, tendons or adjacent tissues, such as the adjacent articular cartilage, can be perforated or otherwise treated to enhance a supply of fluid (e.g. blood) to the area of treatment or repair. The additional availability of fluid, in turn, can assist in resorption of the biocompatible medium and/or the replacement or supplementation thereof with host tissue.

In a representative embodiment comprising insertion into a ligament or tendon of a composition comprising microparticles which have smooth surfaces free from corners and edges and which are suspended in a biocompatible medium, the microparticles induce formation of host tissue (e.g., collagen) at or near the region of insertion. In one example, the microparticles comprise tricalcium hydroxyapatite microspheres, and host collagen is formed around these tricalcium hydroxyapatite microspheres maintaining their position. The addition of this host collagen then gives rise to structural support and stability to, for example, the ACL or PCL deficient knee or ACL/PCL partially deficient knee. Certainly in acute injuries in which the ACL or PCL is being repaired, the addition of the present tissue-generating composition can give rise to increased stability and success for the repair. The smaller ligaments in the carpus and the distal radial ulnar joint and the larger ligaments such as the ACL and PCL are amenable to treatment using the tissue-generating compositions and methods of the present invention, as well.

In accordance with one aspect of the present invention, a composition comprising tricalcium hydroxyapatite microspheres suspended in a sodium carboxymethyl cellulose gel is delivered to a ligament or tendon percutaneously or minimally invasively. In one embodiment, delivering the composition into the tissue of a ligament or tendon comprises injecting the composition into the region of the identified tissue defect. Injecting the composition may comprise using a syringe, as described in greater detail above.

The inserting can comprise inserting a composition into the ligament or tendon while viewing at least a part of the ligament or tendon through a scope. The scope can comprise a video fluoroscope, and the inserting can be fluoroscopically guided. In one implementation, the composition can be impregnated with a water soluble radiopaque dye to facilitate visualization during the inserting of the composition into the ligament or tendon. The radiopaque dye can comprise barium or metrizamide.

In another aspect of the present invention, delivering a composition into the tissue of a ligament or tendon comprises delivering a mesh impregnated with the composition into the tissue. According to one embodiment, the mesh is impregnated with a composition comprising tricalcium hydroxyapatite microspheres suspended in a sodium carboxymethyl cellulose gel. The mesh may be formed in a variety of geometric shapes, with varying thicknesses, lengths, and widths for repair of a particular ligament or tendon. For example, in one embodiment, a cylindrically-shaped mesh impregnated with a composition comprising tricalcium hydroxyapatite microspheres suspended in a sodium carboxymethyl cellulose gel is delivered to an anterior cruciate ligament to treat a defect in the ligament. In another embodiment, a rectangularly-shaped mesh impregnated with compositions described herein is delivered to augment a hernia repair. In still another embodiment, a hemispherical-shaped mesh impregnated with a composition comprising tricalcium hydroxyapatite microspheres suspended in a sodium carboxymethyl cellulose gel is delivered to a ligament or tendon to augment a rotator cuff repair.

Methods of the present invention can augment the repair of a ligament or tendons. The composition can be inserted (e.g., injected) into a ligament or tendon, such as a partial or complete ligament or tendon defect, to thereby facilitate or augment a repair of the ligament or tendon defect. A partial ligament or tendon defect may comprise a ligament or tendon which is not entirely severed. A complete ligament or tendon defect may include for example a ligament which has been completely severed or detached and which, by means known to those skilled in the art, such as, for example, anastomosis using sutures, has been or will be reattached or mended. Thus, in some embodiments, the composition is delivered into a partial tear of a ligament or tendon to help accelerate healing. Similar to prolotherapy techniques, embodiments of the methods described herein can help stiffen a ligament or tendon and provide better stability of the supporting structures.

According to another implementation, a mesh impregnated with a composition comprising tricalcium hydroxyapatite microspheres suspended in a sodium carboxymethyl cellulose is interwoven into a ligament or tendon. For example, an imbrication repair may be undertaken to repair a partially torn tendon. In one embodiment, the repair comprises choosing a mesh from a plurality of sizes, shapes, and geometric forms, thicknesses, heights, widths, and length to repair a ligament or tendon defect in a specific anatomic region. The mesh may be impregnated with a composition, such as tricalcium hydroxyapatite microspheres suspended in a sodium carboxymethyl cellulose, before or after it is chosen or customized for a specific defect and/or anatomic region. In other implementations, the mesh impregnated with the composition is delivered into the tissue of a ligament or tendon through a minimally invasive technique, such as arthroscopically or laparoscopically. In one embodiment, the mesh is delivered to the tissue through an arthroseopie portal or a small incision. The impregnated mesh can enhance an extracapsular repair, such as but not limited to a repair of the medial collateral ligament of the knee, lateral collateral ligament of the knee, or the hip capsule of any mammal. Meshes according to the present invention can also enhance rotator cuff pathology, especially in grade 4 tears that have retracted and are fill thickness. Such tears can be difficult to imbricate and lead to poor results with restrictive range of motion and persistence in pain. According to one embodiment, the shape and dimension of a mesh impregnated with a composition comprising tricalcium hydroxyapatite microspheres suspended in a sodium carboxymethyl cellulose are selected such that, once delivered, the mesh can span a defect in a rotator cuff. The mesh can bridge the gap in the tissue, such that the repair is not placed under significant tension and a better range of motion is possible with a diminution in pain.

Compositions and methods described herein are not limited to spinal disk, ligament, and tendon repair, and encompass a variety of tissue repairs. According to one embodiment of the present invention, a fascial tissue is treated using methods described herein. In one aspect, the method comprises identifying an inguinal, umbilical, or abdominal wall defect and delivering a composition comprising tricalcium hydroxyapatite microspheres suspended in a sodium carboxymethyl cellulose gel into the fascial tissue in the region of the defect. The delivering can comprise delivering a mesh impregnated with the composition into the tissue, or injecting the composition using a syringe.

The above-described embodiments have been provided by way of example, and the present invention is not limited to these examples. Multiple variations and modifications to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. Accordingly, the present invention is not intended to be limited by the disclosed embodiments.

What is claimed is:

1. A method of treating a intervertebral disk comprising:
performing a discography procedure; and
delivering a composition to the invertebral disk through at least one aperture present in the spinal disk created during said discography procedure, wherein the composition comprises tricalcium hydroxyapatite microspheres suspended in a sodium carboxymethyl cellulose gel.

2. The method of claim 1, further comprising:
identifying an annular tear in an intervertebral disk; and
delivering the composition to the intervertebral disk in the region of the annular tear.

3. The method of claim 1, wherein delivering the composition comprises delivering the composition to a nucleus pulposus of the intervertebral disk.

4. The method of claim 2, further comprising at least one of stabilizing the annular tear, reducing loss in the height of the disk, preserving the height of the disk, increasing the height of the disk, increasing a pressure of the disk, improving stability of the disk, and improving structural integrity of the disk.

5. The method of claim 1, wherein the composition comprises approximately 30 percent tricalcium hydroxyapatite microspheres.

6. The method of claim 5, wherein the microspheres have diameters between approximately 25 to 40 microns.

7. The method of claim 2, wherein the annular tear is identified using one of a CT scan, a CT myelography, an MRI scan, and an ultrasound scan.

8. The method of claim 1, wherein delivering the composition comprises viewing the intervertebral disk through a scope.

9. The method of claim 1, wherein the composition comprises a radiopaque material, the method further comprising visualizing the composition by means of the radiopaque material.

10. The method of claim 9, wherein the radiopaque material is metrizamide, barium, or a radial opaque dye.

11. A method of treating a ligament or tendon comprising identifying a defect in the ligament or tendon and delivering a composition into the tissue of the ligament or tendon, wherein the composition comprises tricalcium hydroxyapatite microspheres suspended in a sodium carboxymethyl cellulose gel.

12. The method of claim 11, wherein delivering comprises using a syringe.

13. The method of claim 11, wherein delivering comprises delivering a mesh impregnated with the composition into the tissue.

14. The method of claim 13, wherein the mesh has a generally cylindrical shape.

15. The method of claim 13, wherein the mesh has a generally rectangular shape.

16. The method of claim 13, wherein the mesh has a generally hemispherical shape.

17. The method of claim 13, wherein the mesh is delivered into the tissue arthroscopically or laparoscopically.

18. The method of claim 11, wherein the ligament comprises at least one of an anterior longitudinal ligament, a posterior longitudinal ligament, a supraspinous ligament, an intraspinous ligament, a capsular ligament, an anterior cruciate ligament (ACL), a posterior cruciate ligament (PCL), and a rotator cuff.

19. The method of claim 11, wherein the composition forms a biological scaffold comprising at least a portion of the microspheres, and wherein the biological scaffold operates at least as partial connective tissue in the ligament or tendon.

20. A method of treating a fascial tissue comprising:
identifying an inguinal, umbilical, or abdominal wall defect; and
delivering a composition into the fascial tissue in the region of the defect, wherein the composition comprises tricalcium hydroxyapatite microspheres suspended in a sodium carboxymethyl cellulose gel.

21. The method of claim 20, wherein delivering comprises delivering a mesh impregnated with the composition into the tissue.

22. The method of claim 20, wherein delivering comprises using a syringe.

23. A method of treating an intervertebral disk comprising:
inserting one or more needles into the intervertebral disk creating one or more apertures in the intervertebral disk;
injecting contrasting die into the intervertebral disk; and
delivering a composition to the intervertebral disk through at least one of said one or more apertures, wherein the composition comprises tricalcium hydroxyapatite microspheres suspended in a sodium carboxymethyl cellulose gel.

24. A method of treating an intervertebral disk comprising placing a plurality of tricalcium hydroxyapatite particles suspended in a sodium carboxymethyl cellulose gel into an interior portion of the intervertebral disk.
